(19) United States
(12) Patent Application Publication
(10) Pub. No.: US 2014/0046325 A1
(43) Pub. Date: Feb. 13, 2014

(54) DISPOSABLE ELECTROTHERMAL CAUTERIZATION AND LIGATION TOOL

(71) Applicants: David Weber, Bloomington, IN (US); Thomas P. Clement, Bloomington, IN (US)

(72) Inventors: David Weber, Bloomington, IN (US); Thomas P. Clement, Bloomington, IN (US)

(21) Appl. No.: 13/569,667

(22) Filed: Aug. 8, 2012

(51) Int. Cl. A61B 18/14

(52) U.S. Cl. CPC A61B 18/1447
USPC 606/51

(57) ABSTRACT

A disposable device for tissue cauterization, including a generally electrically insulating housing assembly and a generally electrically conducting elongated assembly. The housing assembly includes a barrel having a cylindrical aperture formed therethrough, a grip extending from the barrel, a trigger slidingly connected to the barrel, a spring connected to the trigger, and an end plug positioned adjacent the proximal end of the barrel. The elongated assembly includes a hollow elongated outer cylinder disposed within the barrel and extending from the distal end, a generally hollow elongated inner cylinder slidably disposed within the elongated outer cylinder and extending to snugly engage the end plug, and bipolar forceps operationally connected to the distal end of the elongated inner cylinder and defining a leaf spring. Actuation of the trigger urges the inner cylinder to slide in a proximal direction relative to the outer cylinder, urging the bipolar forceps partially into the outer cylinder and closer together.

### BACKGROUND

Blood loss is a problem during surgery. One way of addressing the issue of bleeding is to sew or suture shut ruptured blood vessels. However, this method is time-consuming and is not always practical, especially for small or hard to reach ruptures. Another method for stopping bleeding is cauterization. When heat is properly applied to tissue, the wound may be sealed and bleeding stopped. Prior to modern surgical devices, a heated pieced of metal was used to accomplish cauterization. This technique often led to rudimentary results, such as scarring and imprecise handling of tissue. Additionally, infection was a risk.

One advance in cauterization and tissue ligation has been the development of electrothermal devices for quickly and precisely generating and applying heat. Of these, the Kleppinger has become the standard for tissue cauterization. The Kleppinger, however, still has its shortcomings. First, the Kleppinger is awkward in its design, having a gripping and actuation mechanism that is counterintuitive. The Kleppinger requires the operator to grip the tool by engaging a thumb-ring with the thumb and a gripping wedge between the first and second fingers. To actuate the cauterization tip against tissue, it is necessary to hold the first and second fingers steady while moving the thumb ring forward. However, the more intuitive actuation gesture is to pull the first and second fingers back, moving the wedge back towards the thumb ring. However, this gesture pulls the cauterization tip away from the tissue to which it has been engaged. Improper use in this manner leads to disengagement of the cauterization tip from the target tissue which results in imprecise targeting of tissue and can result in unintended damage to surrounding tissue, thus harming the patient.

Further, the Kleppinger is expensive and accordingly is designed to be reusable. Thus, the Kleppinger must be autoclaved under high temperatures and intense pressure for sterilization. Repeatedly subjecting the Kleppinger device to such extreme temperature and pressure conditions degrades its electrical connections and leads to unreliable use of the device. Accordingly, the operator (i.e. the surgeon) of the device is typically inconvenienced and irritated when the device malfunctions, and this ultimately puts the patient at risk.

Thus, there remains a need for a more reliable, intuitive, precise and inexpensive electrothermal device for tissue cauterization and ligation. The present invention addresses these needs.

### BRIEF DESCRIPTION OF THE DRAWINGS

FIG. 1 is a perspective view of a ligation device according to a first embodiment of the novel technology.

FIG. 2 is a side elevation view of the embodiment of FIG. 1.

FIG. 3 is an enlarged side view of the handle assembly portion of the embodiment of FIG. 1.

FIG. 4 is a side skeletal view of the embodiment of FIG. 1.

FIG. 5 is a perspective view of of the embodiment of FIG. 1.

FIG. 6 is a top plan view of the embodiment of FIG. 1.

FIG. 7 is a perspective view of the embodiment of FIG. 1.

FIG. 8 is a process control chart of a method for cauterizing tissue with an embodiment of the device.

### DETAILED DESCRIPTION OF THE EMBODIMENTS

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated device and such further applications of the principles of the novel technology as illustrated therein being contemplated as would normally occur to one skilled in the art to which the novel technology relates.

The present novel technology relates to a single use or ‘disposable’ device 10 for electrothermally inducing tissue coagulation. The device 10 includes an electrically insulating handle assembly 20 and an elongated assembly 30 which is anchored in the handle assembly 20 and extends therefrom in a distal direction. The distal direction is the direction extending away from the handle assembly 20. The proximal direction is the direction extending toward the handle assembly 20.

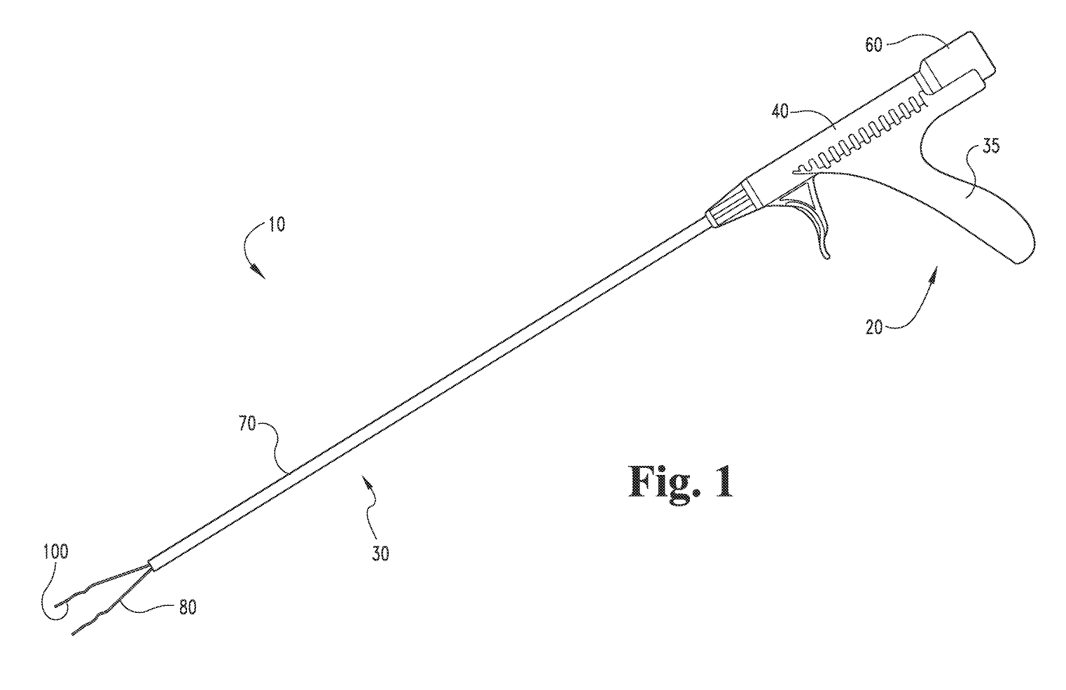

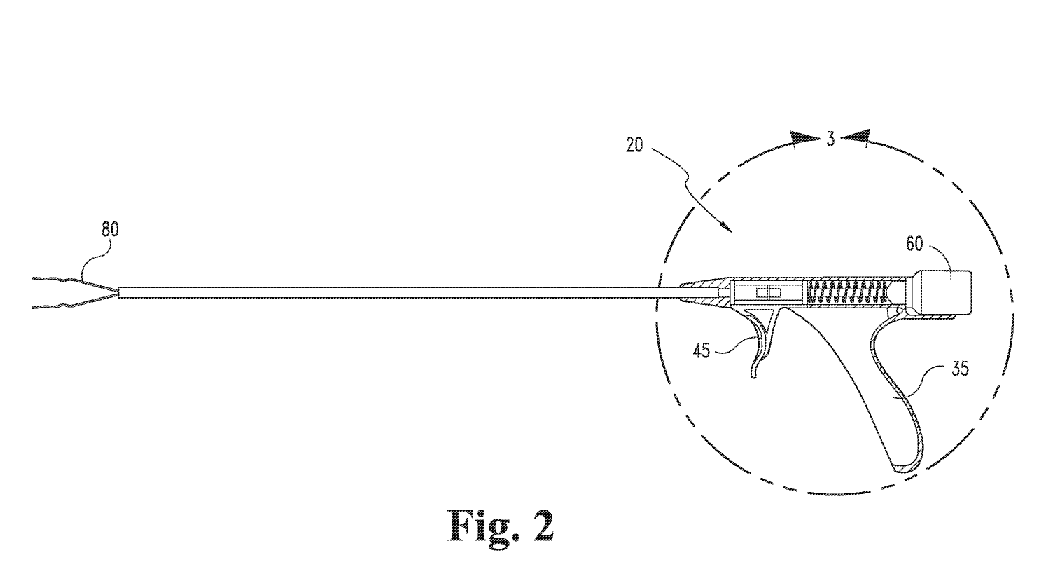

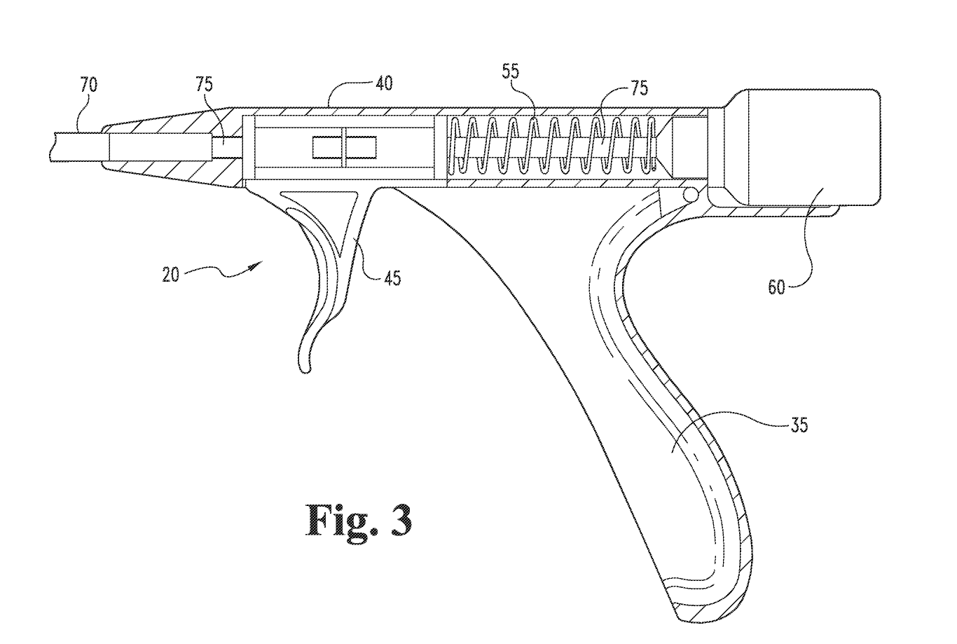

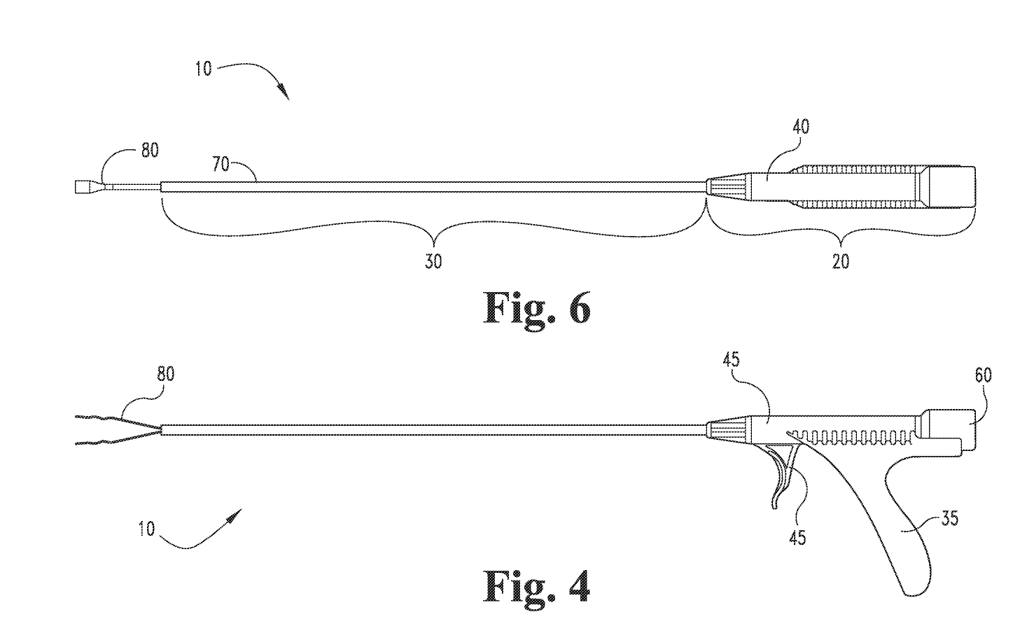

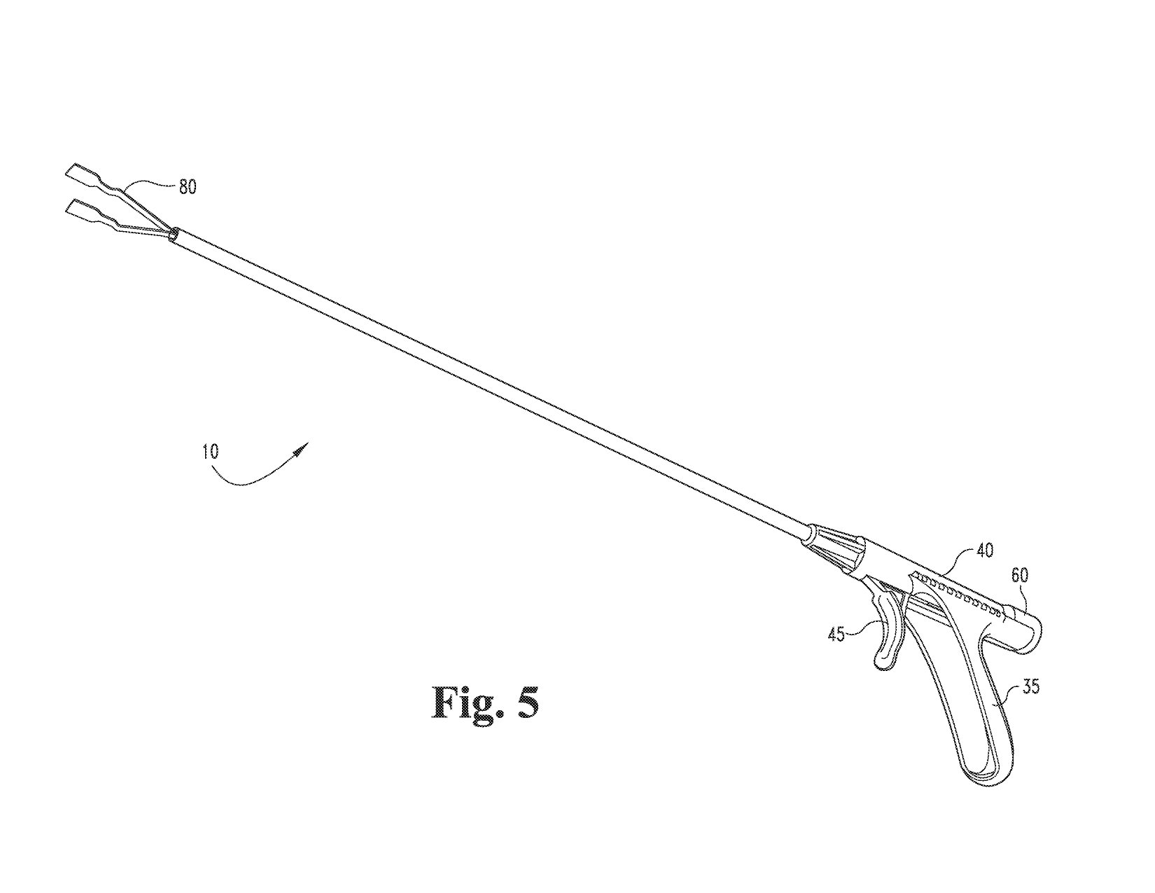

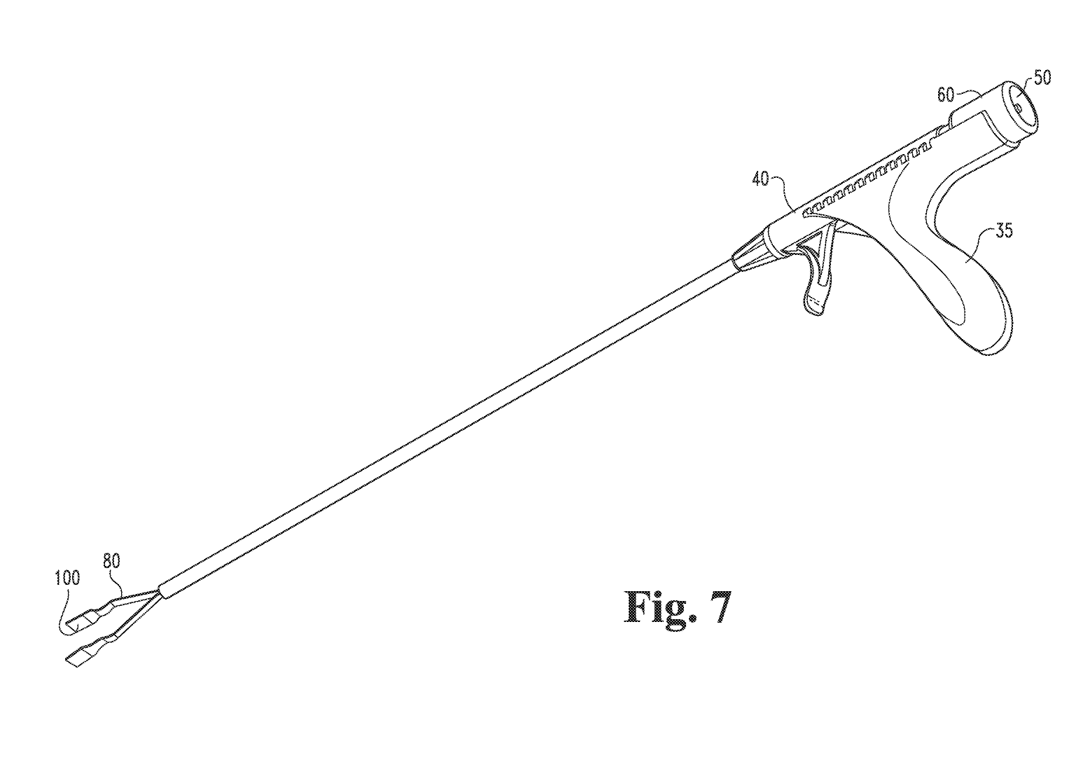

As shown if FIGS. 1-7, the handle assembly 20 includes a pistol grip portion 35 connected to and extending from a barrel portion 40, from which a trigger portion 45 likewise extends. These portions 35, 40, 45 are typically formed from a lightweight, electrically insulating material, such as a rigid plastic, rubber, fiberglass or other suitable materials. The barrel portion 40 includes a generally cylindrical aperture 50 formed therethrough, sized to snugly engage the elongated extension assembly 30. A tension member 55, such as a spring or other suitable member, is positioned in the barrel portion 40 and operationally connected to the trigger portion 45 to urge the trigger portion 45 in the distal direction relative to the barrel portion 40. An end plug 60 is positioned adjacent and rotatably connected to the opposite, proximal end of the barrel portion 40.

The elongated assembly 30 includes an elongated outer cylinder 70 and an elongated inner cylinder 75 sized to be slidably positioned in and extend through the elongated outer cylinder 70. The elongated outer cylinder 70 is positioned to partially extend through the barrel portion 40, with the remainder extending from the distal end of the barrel portion 40. The elongated inner cylinder 75 extends from the proximal end of the elongated outer cylinder 70 and through the end plug 60, to which it is lockingly engaged. Rotation of the end plug 60 likewise rotates the inner cylinder 75 around a major axis extending therethrough. A pair of elongated generally flat electrically conductive paddles 80 or bipolar forceps are operationally connected to and extend from the distal end of the elongated inner cylinder 75, and are positioned to likewise extend from the elongated outer cylinder 70. The respective flat paddles 80, which may also be thought of as forceps and/or electrodes, typically define a leaf spring, the leaves of which are urged apart by tensile or spring forces unless otherwise acted upon. The paddles 80 may be coated with a highly electrically conductive material 100, typically having a conductivity of at least 9.43×10⁷ S/m at 20° C., such as platinum, gold, silver, or other suitable materials.

In operation, actuation of the trigger portion 45 urges the inner cylinder 75 to travel in a proximal direction relative to the stationary outer cylinder 70, partially engaging and retracting the pair of elongated flat paddles 80 into the outer cylinder 70. Such retraction of the paddles 80 generates a force counter to the tensile force urging the paddles 80 apart, and thus the paddles 80 are urged together when the trigger portion 45 is actuated.

In an alternate embodiment, the flat paddles 80 may lack rotational capabilities and the end plug 60 may not be incorporated. Instead, a non- rotational plug 65 would be connected in place of end plug 60 in a substantially similar position as the end plug 60.

In another embodiment, the distal end of the elongated extension assembly 30 may incorporate a pair of generally ridged electrically conductive paddles. The pair of generally ridged electrically conductive paddles may be made of steel, typically surgical grade steel, or other suitable material. The ridged electrode paddles may be coated with a highly electrically conductive material, typically having a conductivity of at least 9.43×10⁷ S/m at 20° C., such as platinum, gold, silver or other suitable material.

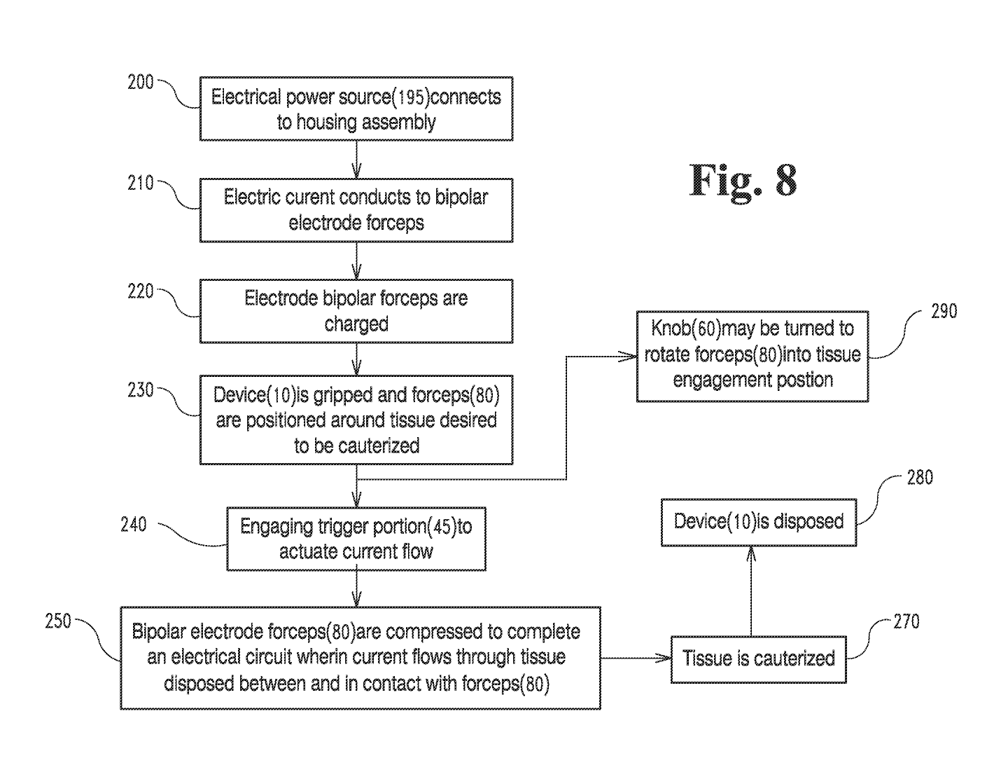

As shown in FIG. 8, an electric power source 195 is operationally connected 200 to an electrical connection portion 205 disposed within the housing assembly 20, typically within plug 60. The electric power source 200 is connected in electric communication with forceps 80. In operation current conducts 210 through the elongated extension assembly 30 and electrically charges 220 the electrode forceps 80 coated with a highly conductive material 100. The device 10 is gripped 230 at the insulating handle assembly 20, and the forceps are positioned around tissue to be cauterized. The trigger portion 45 is engaged 240, which urges the flat paddles 80 together 250. The knob portion 60 may be rotated to yield rotation 290 of the flat paddles 80 by a substantially similar magnitude. Tissue cauterizes 270 on contact with the flat electrode paddles 80. After a desired amount of cauterization, the device is discarded 280.

In some embodiments, the trigger portion 45 is lockably engagable, wherein the trigger portion 45 may be engaged and locked, causing the flat paddles 80 to remain compressed without constant force applied to on the trigger portion 45.

Referring now to another embodiment, the knob portion 60 is operationally connected to the flat paddles 80 and rotation of the knob portion 60 actuates rotation of the electrode paddles 80 in the opposite direction.

In some embodiments, the electrically conductive elongated assembly 30 is easily detachable from the electrically nonconductive handle assembly 30. The electrically conductive elongated assembly 30 may be recyclable in alternate embodiments, whereby the electrically conductive elongated assembly 30 may be removed from the device and magnetically separated, de-tinned, melted, and reformed or by other suitable processes while the handle assembly 20 is retained for reuse. A new elongated assembly 30 may then be connected to the handle assembly 20.

The inner and outer cylinders 75, 70 and electrode paddles 80 may be made from an electrically and thermally conductive structural material, such as steel, and, more typically, surgical grade steel. The electrode paddles 80 may be plated with a highly electrically conductive material 100, such as gold or other suitable materials.

In some embodiments, a ratchet portion is operationally connected to the housing assembly 20, such that the trigger portion 45 may be partially actuated to partially retract the paddles 80, thus moving them a desired distance apart; the ratchet portion engages to prevent the spring 55 from engaging the inner cylinder 75 to urge it in a distal direction and restore it to its previous distal position, thus urging the paddles 80 from the distal end of the outer cylinder 75 and allowing the leaf spring to urge the paddles 80 apart. The ratchet may automatically engage to hold the trigger portion 45 in place relative to the barrel portion 40 and thus maintain a desired distance between the paddles 80. Disengagement of the ratchet allows the spring 55 to restore the paddles 80 to their extended and separated orientation.

Electrical connection may be made to the proximal end of the elongated inner cylinder 75 to power the device.

In some embodiments, the end plug 60 may be rotated; such rotation likewise rotates the paddles 80 relative the housing assembly 20.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

What is claimed is:

1) A disposable device for tissue cauterization, comprising:
a generally electrically insulating housing assembly; and
a generally electrically conducting elongated assembly operationally connected to the housing assembly;
wherein the housing assembly further comprises:
a barrel portion having a proximal end, a distal end, and a cylindrical aperture formed therethrough;
a grip portion extending from the barrel portion;
a trigger portion slidingly connected to the barrel portion;
a spring portion operationally connected to the trigger portion and the barrel portion to urge the trigger portion in a distal direction; and
an end plug portion positioned adjacent the proximal end of the barrel portion; and
wherein the elongated assembly further comprises:
a generally hollow elongated outer cylinder disposed within the barrel portion and extending from the distal end;
a generally hollow elongated inner cylinder substantially slidably disposed within the elongated outer cylinder and extending from the proximal end thereof to penetrate and snugly engage the end plug;
bipolar forceps operationally connected to the distal end of the elongated inner cylinder and defining a leaf spring;
wherein actuation of the trigger portion urges the inner cylinder to slide in a proximal direction relative to the outer cylinder, urging the bipolar forceps partially into the outer cylinder and closer together.

2) The device of claim 1, wherein the end plug may be rotated relative the housing assembly; and wherein rotation of the end plug rotates the bipolar forceps relative the housing assembly.

3) The device of claim 1, wherein the trigger portion is actuatable to urge the bipolar forceps together.

4) The device of claim 1, wherein the inner cylinder is operationally connected to an electrical power source in electric communication with the bipolar forceps.

5) The device in claim 1 wherein the trigger portion may be lockingly engaged to hold the bi-polar forceps in a position of partial compression.

6) The device of claim 1, wherein the forceps are ridged for increased gripping.

7) The device of claim 1 wherein the bipolar forceps are gold plated.

8) A method for cauterizing tissue comprising:
a) operationally connecting an electrical power source to cauterization device having a generally electrically insulating handle portion connected to a generally electrically conducting elongated assembly;
b) gripping the handle portion;
c) positioning a pair of electrically conductive paddles extending from the generally electrically conducing elongated assembly around tissue to be cauterized;
d) engaging a trigger portion to urge together the pair of electrically conducting paddles extending from the elongated stem assembly;
e) energizing the electrically conducting paddles to cauterize tissue; and
f) discarding the electrically conducting paddles and the elongated assembly after tissue has been cauterized.

9) The method of claim 8 wherein the trigger portion is lockably engagable to yield constant compression of the pair of bipolar forceps.

10) The method of claim 8 further comprising rotating a knob portion, wherein rotation of the knob portion causes rotation of the pair of bipolar forceps.

11) The method of claim 8 wherein the pair of bipolar forceps is coated with a highly electrically conductive material.

12) A single use tubal ligation device, comprising:
an electrically conductive elongated assembly operationally connected to an electrically nonconductive handle assembly;
a trigger portion operatively connected to the electrically nonconductive handle assembly;
an electrical connection disposed within an end plug operatively connected to the electrically nonconductive hand assembly;
a pair of forceps operationally connected to the electrically conductive elongated assembly;
wherein the electrical connection may be actuated to electrically energized the pair of bipolar forceps;
a tension member disposed within the electrically nonconductive handle assembly and operationally connected to the trigger portion to urge the trigger portion in a distal direction;
wherein movement of the trigger portion opposite the distal direction urges the bipolar forceps together; and
wherein the pair of bipolar forceps are coated with a highly electrical conductive material.

13) The device of claim 12, wherein the elongated assembly further comprises an inner cylinder slidingly disposed within an outer cylinder

14) The device of claim 12, wherein the knob portion actuates rotation of the bipolar forceps.

15) The device of claim 12, wherein the trigger portion is lockingly engagable to actuate and compress the bipolar forceps.

16) The device of claim 12 wherein the electrical connection conducts electricity to the bipolar forceps.

17) The device of claim 12 wherein the electrically nonconductive trigger portion is a pistol trigger.

18) The device of claim 12 wherein a knob portion is operationally connected to the handle assembly.

19) The device of claim 12 wherein the tension member is a spring.

20) The device of claim 12 wherein the bipolar forceps define a leaf spring urging the respective forceps away from each other.
